(19) United States
(12) Patent Application Publication
(10) Pub. No.: US 2025/0262404 A1
(43) Pub. Date: Aug. 21, 2025

(54) INTERACTIVE REHABILITATION SYSTEM

(71) Applicant: CHANG GUNG UNIVERSITY, Taoyuan City (TW)

(72) Inventors: Shih-Ying CHIEN, Taoyuan City (TW); Ching-Yi WU, Taoyuan City (TW)

(21) Appl. No.: 18/820,846

(22) Filed: Aug. 30, 2024

(30) Foreign Application Priority Data
Feb. 21, 2024 (TW) 113106161

(51) Int. Cl. A61M 21/00; G06F 3/039; G06F 3/041

(52) U.S. Cl. CPC A61M 21/00; G06F 3/0393; G06F 3/0416; A61M 2021/005; A61M 2205/505; A61M 2205/584

(57) ABSTRACT

An interactive rehabilitation system includes an electronic device, a question board and an operating piece. The electronic device includes a processing unit and a touchscreen. The question board is configured to be detachably placed on the touchscreen, and includes a board body that is formed with a plurality of through holes. The operating piece has a cross-sectional shape that matches a cross-sectional shape of one of the through holes. The touchscreen is configured to detect a contact from the operating piece, and to transmit a touch signal to the processing unit when the operating piece is inserted through one of the through holes, when the operating piece is in contact with the touchscreen, and when the touchscreen detects a contact from the operating piece. The processing unit is configured to generate and record operation data based on the touch signal thus received.

### CROSS-REFERENCE TO RELATED APPLICATION

This application claims priority to Taiwanese Invention Patent Application No. 113106161, filed on Feb. 21, 2024, the entire disclosure of which is incorporated by reference herein.

### FIELD

The disclosure relates to a rehabilitation system, and more particularly to an interactive rehabilitation system.

### BACKGROUND

Stroke patients often experience visual perception and motor coordination problems, which makes it difficult for them to resume their daily activities. However, in 1992, Richard J. Davidson proposed that for patients with brain injuries caused by diseases or external factors, the structure and function of the cerebral cortex may be improved through repeated stimulation on the non-necrotic neurons. This phenomenon is known as neuro-plasticity, or brain plasticity. In other words, training for strengthening an affected limb of a patient can help activate the non-necrotic neurons in the cerebral cortex around the lesion. Through appropriate rehabilitation training and repeated practice, patients can “reorganize the nervous system”, thus improving the limb function (Nudo, 2003). Furthermore, the smaller the damaged part of the brain, the easier and faster the recovery. Therefore, prescribing accurate and appropriate limb rehabilitation training is considered one of the key elements for enabling stroke patients to resume their daily activities. However, conventional rehabilitation is labor-intensive. Rehabilitation therapists often conduct rehabilitation training and assessment in a time-consuming and laborious one-to-one manner. Conventional rehabilitation also lacks detailed records of patients' movements and training sessions, as well as comprehensive tracking of patients' recovery profiles.

### SUMMARY

Therefore, an object of the disclosure is to provide an interactive rehabilitation system that can alleviate at least one of the drawbacks of the prior art.

According to the disclosure, the interactive rehabilitation system includes an electronic device, a question board and an operating piece.

The electronic device includes a processing unit, and a touchscreen that is electrically connected to the processing unit. The question board is configured to be detachably placed on the touchscreen, and includes a board body that is formed with a plurality of through holes. The operating piece has a cross-sectional shape that matches a cross-sectional shape of one of the through holes. The touchscreen is configured to detect a contact from the operating piece, and to transmit a touch signal to the processing unit when the operating piece is inserted through one of the through holes that has the cross-sectional shape matching the cross-sectional shape of the operating piece, when the operating piece is in contact with the touchscreen, and when the touchscreen detects a contact from the operating piece. In response to receipt of the touch signal, the processing unit is configured to generate and record operation data based on the touch signal thus received. The operation data includes a location where the operating piece is in contact with the touchscreen and a time length of time it takes to place the operating piece.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating an interactive rehabilitation system according to a first embodiment of the present disclosure.

FIG. 2 is a perspective view of the interactive rehabilitation system according to an embodiment of the present disclosure.

FIG. 3 is a schematic view of the interactive rehabilitation system illustrating a first question board placed on an electronic device.

FIG. 4 is a schematic view of the interactive rehabilitation system illustrating a second question board placed on the electronic device.

FIG. 5 is a schematic view of the interactive rehabilitation system illustrating a third question board placed on the electronic device.

FIG. 6 is a schematic view illustrating a touchscreen of the electronic device displaying operating options to be selected by a user.

FIG. 7 is a schematic view illustrating touch patterns on back sides respectively of the first, second and third question boards according to an embodiment of this disclosure.

FIG. 8 is a schematic view illustrating the touch patterns according to another embodiment of this disclosure.

FIG. 9 is a block diagram illustrating the interactive rehabilitation system according to a second embodiment of the present disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

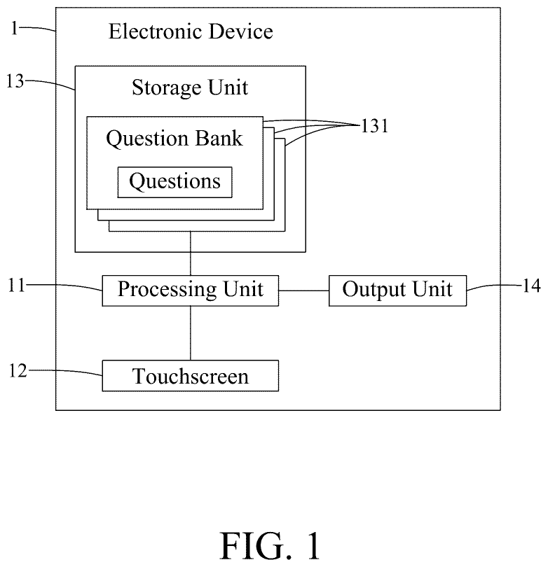

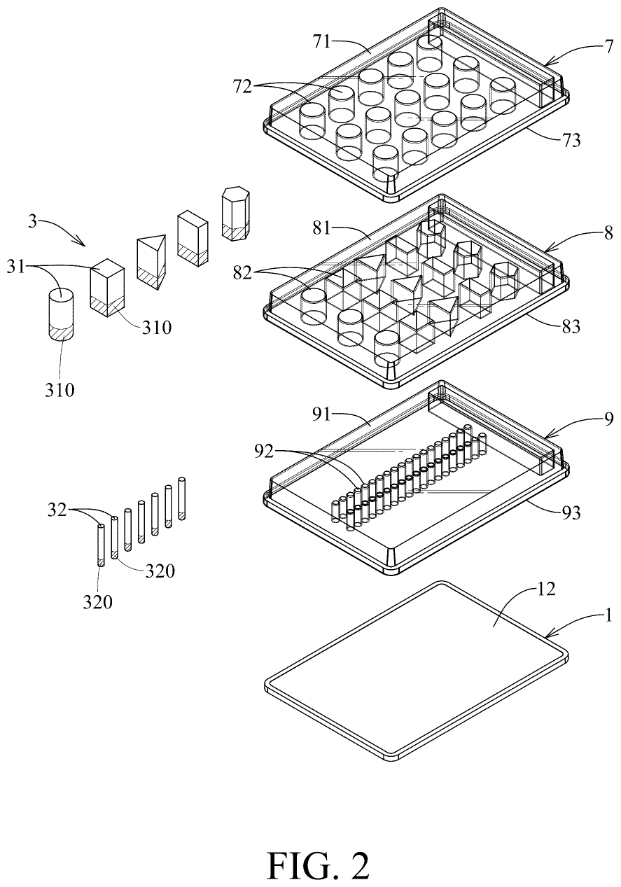

Referring to FIGS. 1 and 2, an interactive rehabilitation system according to a first embodiment of this disclosure is presented. In this embodiment, the interactive rehabilitation system includes an electronic device 1, three question boards 7, 8, 9 and a set of operating pieces 3.

The electronic device 1 includes a processing unit 11 (e.g., a microprocessor, a central processing unit, etc.), a touchscreen 12 that is electrically connected to the processing unit 11, a storage unit 13 (e.g., a hard disk drive, a random-access memory (RAM) device, etc.), and an output unit 14 (e.g., a speaker). For example, the electronic device 1 may be embodied as, but is not limited to, a tablet (e.g., an iPad®), a smartphone, or any device that has computing capability and a touchscreen.

Each of the question boards 7, 8, 9 is configured to be detachably placed on the touchscreen 12 (i.e., only one of the question boards 7, 8, 9 is placed on the touchscreen 12 at a time of use), and is made of a transparent material. A first one of the question boards 7 (hereinafter referred to as “the first question board”) includes a first board body 71 that is formed with a plurality of first through holes 72. Each of the first through holes 72 has a cross-sectional shape of a circle and has a first inner diameter. A second one of the question boards 8 (hereinafter referred to as “the second question board”) includes a second board body 81 that is formed with a plurality of second through holes 82. The second through holes 82 have various cross-sectional shapes that include circles, squares, triangles, rectangles, and hexagons, and there are multiple second through holes 82 for each shape. A third one of the question boards 9 (hereinafter referred to as “the third question board”) includes a third board body 91 that is formed with a plurality of third through holes 92. A cross-sectional shape of each of the third through holes 92 is also a circle, and each of the third through holes 92 has a second inner diameter that is much smaller than the first inner diameter of each of the first through holes 72.

Each operating piece in the set of operating pieces 3 has a cross-sectional shape that matches the cross-sectional shape of one or more of the first through holes 72, the second through holes 82, and the third through holes 92. In this embodiment, the set of operating pieces 3 includes a plurality of first operating pieces 31, and a plurality of second operating pieces 32. The first operating pieces 31 have cross-sectional shapes of a circle, a square, a triangle, a rectangle, and a hexagon. One of the first operating pieces 31 has the cross-sectional shape that matches the cross-sectional shapes of the first through holes 72. Each of the first operating pieces 31 has the cross-sectional shape that matches the cross-sectional shape of at least one of the second through holes 82. Each of the second operating pieces 32 has a cross-sectional shape that is a circle and that matches the cross-sectional shapes of the third through holes 92. Each of the first and second operating pieces 31, 32 has an end portion 310, 320 that includes a conductive material. In this embodiment, the first and second operating pieces 31, 32 are exemplified as pillars. As shown in FIG. 2, the first operating pieces 31 are exemplified as a cylinder, a square prism, a triangular prism, a rectangular prism and a hexagonal prism, and each of the second operating pieces 32 is exemplified as a slender cylinder (stick).

The storage unit 13 stores a plurality of question banks 131 respectively for the question boards 7, 8, 9. In this embodiment, a number of the question banks 131 is three. The question banks 131 are related respectively to the question boards 7, 8, 9. Each of the question banks 131 includes a plurality of questions. For example, each of the questions may be related to, but is not limited to, displaying a color at a location on the touchscreen 12, and the processing unit 11 is configured to control the touchscreen 12 to display the color at the location on the touchscreen 12 according to the question. For a question of one of the question banks 131 that is related to the first question board 7, the location on the touchscreen 12 corresponds to a position of one of the first through holes 72 of the first question board 7. Similarly, for a question of one of the question banks 131 that is related to the second question board 8, the location on the touchscreen 12 corresponds to a position of one of the second through holes 82 of the second question board 8; for a question of one of the question banks 131 that is related to the third question board 9, the location on the touchscreen 12 corresponds to a position of one of the third through holes 92 of the third question board 9. In some embodiments, the processing unit 11 is further configured to control the output unit 14 to instruct (e.g., through a voice message or music) a user to insert one operating piece 31, 32 into one of the first through holes 72, the second through holes 82, and the third through holes 92 where the touchscreen 12 is displaying the color. Specifically, said one operating piece 31, 32 is selected from among the set of operating pieces 3 and has the cross-sectional shape matching the cross-sectional shape of said one of the first through holes 72, the second through holes 82, and the third through holes 92.

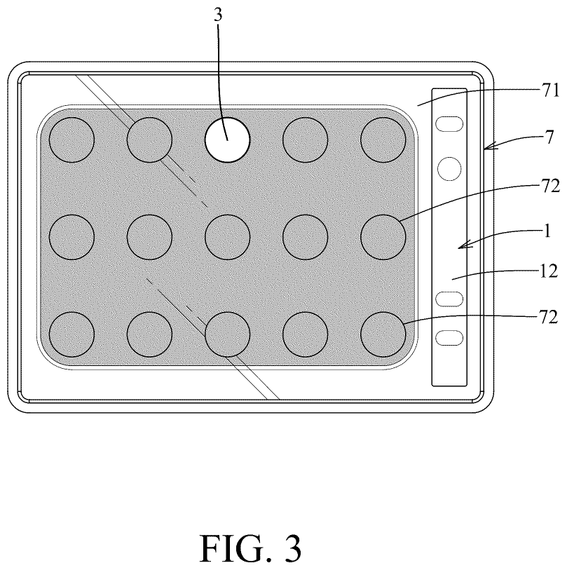

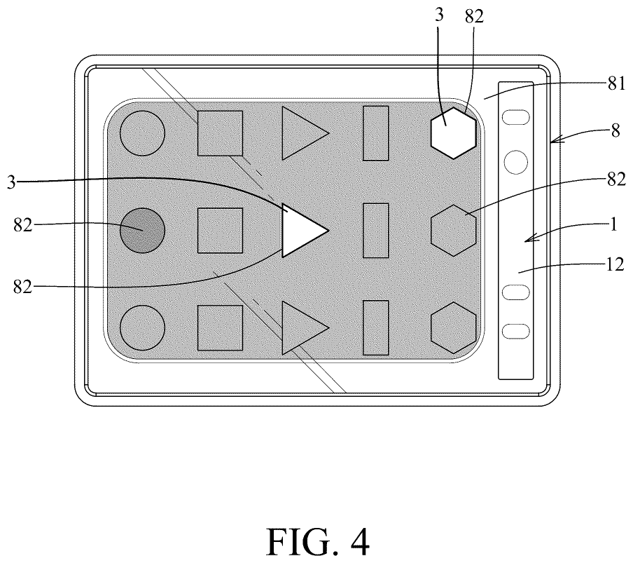

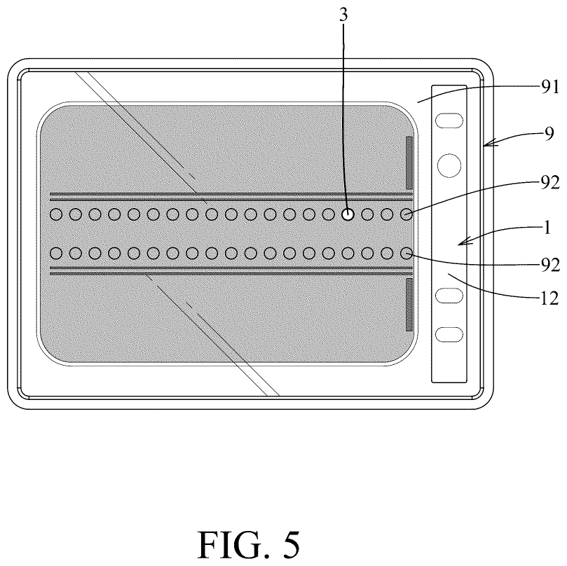

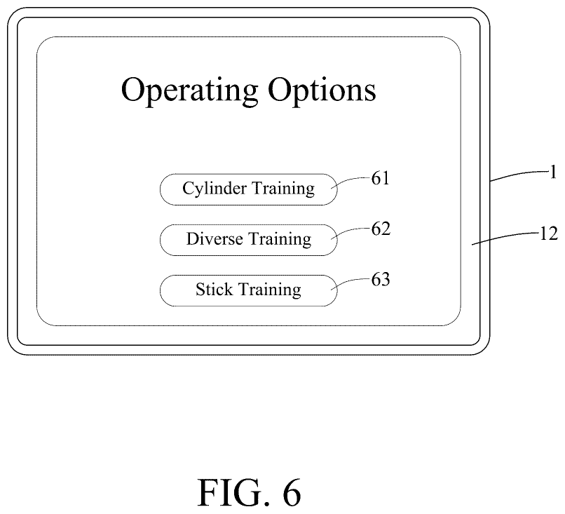

Referring to FIG. 6, in this embodiment, the processing unit 11 (see FIG. 1) first controls the touchscreen 12 to display three operating options that are associated respectively with the question banks 131 and that correspond respectively to the question boards 7, 8, 9. When a user wants to use the interactive rehabilitation system to perform upper limb (e.g., an arm) rehabilitation training, the user may select one of the operating options that is displayed on the touchscreen 12 of the electronic device 1. The operating options may include, for example, a first option 61 for cylinder training that corresponds to the first question board 7 (see FIG. 3), a second option 62 for diverse training that corresponds to the second question board 8 (see FIG. 4), and a third option 63 for stick training that corresponds to the third question board 9 (see FIG. 5). For the sake of brevity, only operations related to a selection of the second option 62 corresponding to the second question board 8 will be described in detail in the rest of this disclosure.

Once the user selects the second option 62, and the processing unit 11 determines that the second option 62 has been selected, the processing unit 11 obtains, from the storage unit 13, the question bank 131 that is associated with the second option 62 and that is related to the second question board 8. Then, the user may place the second question board 8 on the touchscreen 12.

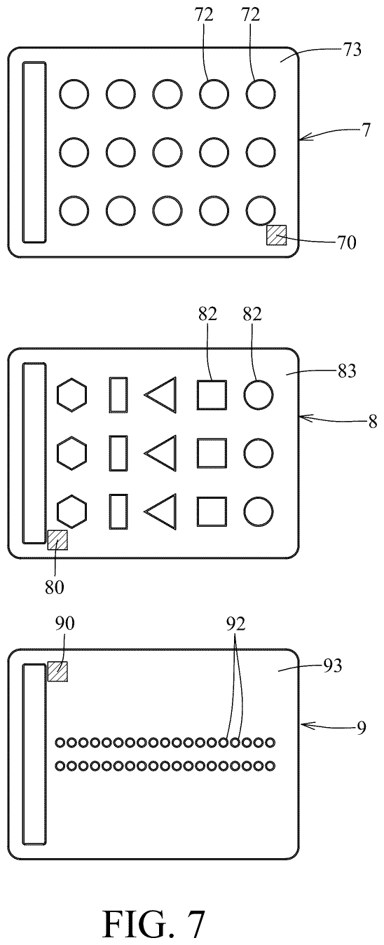

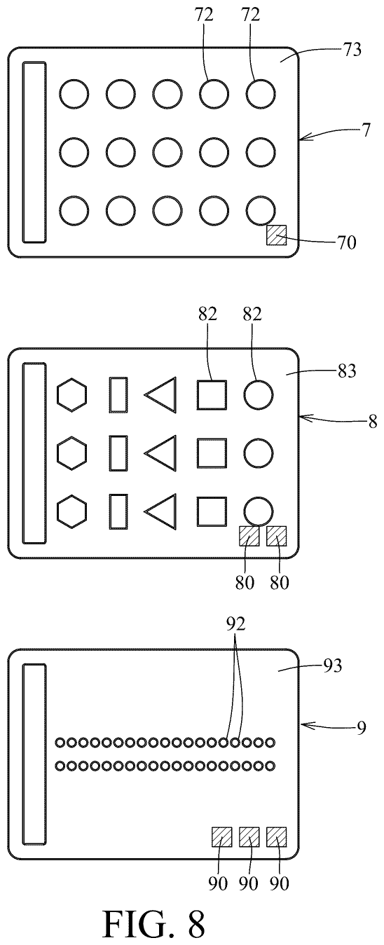

In other embodiments, each of the first, second and third question boards 7, 8, 9 further includes a back side 73, 83, 93 formed with a touch pattern 70, 80, 90 (see FIGS. 7 and 8) that is associated with a corresponding one of the question banks 131, and that includes a conductive material. In one embodiment as shown in FIG. 7, each of the touch patterns 70, 80, 90 respectively of the question boards 7, 8, 9 is exemplified as a film marker that is made of a conductive material and the touch patterns 70, 80, 90 are located at different locations on the first, second and third question boards 7, 8, 9. For example, the touch pattern 70 of the first question board 7 is located at a bottom right corner of the back side 73 of the first question board 7, the touch pattern 80 of the second question board 8 is located at a bottom left corner of the back side 83 of the second question board 8, and the touch pattern 90 of the third question board 9 is located at a top left corner of the back side 93 of the third question board 9. In another embodiment as shown in FIG. 8, the touch patterns 70, 80, 90 may be located on the back sides 73, 83, 93 at corresponding locations (e.g., the bottom right corners of the back sides 73, 83, 93 of the question boards 7, 8, 9), and the touch patterns 70, 80, 90 of the question boards 7, 8, 9 have different numbers of film markers. For example, the touch pattern 70 of the first question board 7 has only one film marker, the touch pattern 80 of the second question board 8 has two film markers, and the touch pattern 90 of the third question board 9 has three film markers. In the embodiments where the first, second and third question boards 7, 8, 9 include the touch patterns 70, 80, 90, the touchscreen 12 transmits a pattern signal that corresponds to the touch pattern 70, 80, 90 of a corresponding one of the question boards 7, 8, 9 to the processing unit 11 when the touchscreen 12 detects the touch pattern 70, 80, 90 of the corresponding one of the question boards 7, 8, 9 that is placed on the touchscreen 12. The processing unit 11, in response to receipt of the pattern signal, obtains, from the storage unit 13, one of the question banks 131 that is related to the corresponding one of the question boards 7, 8, 9 according to the pattern signal. Specifically, the processing unit 11 is configured to analyze the pattern signal to determine which one of the first, second and third question boards 7, 8, 9 that has the touch pattern 70, 80, 90 corresponding to the pattern signal is placed on the touchscreen 12 now, and then to obtain the question bank 131 related to the question board 7, 8, 9 that is determined to be placed on the touchscreen 12 now.

The processing unit 11, after obtaining the question bank 131 that is associated with the second option 62 from the storage unit 13, controls the touchscreen 12 to display the questions of the question bank 131 thus obtained one by one. For example, as shown in FIG. 4, the processing unit 11 controls, according to one of the questions, the touchscreen 12 to display a color (e.g., red) at three locations on the touchscreen 12 that correspond respectively to three of the second through holes 82 at the following locations: a rightmost column in the first row, a leftmost column in the second row, and a middle column in the second row. In other words, the second question board 8 with the aforementioned three of the second through holes 82 showing the color may be seen as a question to be answered by the user, and the content of the question is the aforementioned locations where the touchscreen 12 displays the color. In other embodiments, the touchscreen 12 may display different colors (e.g., yellow, blue, and red) at the aforementioned locations that correspond to the three of the second through holes 82 and may request that the user insert three of the first operating pieces 31 one by one according to a predetermined sequence (e.g., first blue, then yellow, and finally red).

When the touchscreen 12 displays the question, the processing unit 11 controls the output unit 14 to emit a verbal instruction (e.g., “please insert the operating pieces into the columns in red.”), in order to instruct the user to insert the operating pieces 31, 32 into the second through holes 82 where the color is being shown. After the processing unit 11 controls the touchscreen 12 to display a first one of the questions included in the question bank 131, the processing unit 11 starts a timer.

When one of the operating pieces 31, 32 is inserted into one of the second through holes 82 and is in contact with the touchscreen 12, the touchscreen 12 detects a contact of the one of the operating pieces 31, 32 and transmits a touch signal to the processing unit 11. In response to receipt of the touch signal, the processing unit 11 generates and records operation data based on the touch signal thus received. The operation data includes a location where the one of the operating pieces 31, 32 is in contact with the touchscreen 12 (hereinafter referred to as “the touch location”) and a time length of the time it takes the user to place the one of the operating pieces 31, 32 (i.e., between the user being requested to select one of the operating pieces 31, 32 that is to be inserted into one of the second through holes 82 and the touchscreen 12 having detected the contact of the one of the operating pieces 31, 32) (hereinafter referred to as “the answering time”).

Once the timer has been started, the processing unit 11 generates and records operation data each time the processing unit 11 receives a touch signal from the touchscreen 12 until the processing unit 11 determines that the timer has counted to a first predetermined time length of, for example, but not limited to, five minutes. That is to say, the user may continuously make attempts (i.e., selecting one of the operating pieces 31, 32 and inserting the selected one of the operating pieces 31, 32 into one of the second through holes 82) to answer the question currently displayed on the touchscreen 12, and the user may continue to answer as many questions in the question bank 131 as possible (i.e., related to the second question board 8) before the timer counts to the first predetermined time length. When the timer has counted to the first predetermined time length, the processing unit 11 stops receiving touch signals from the touchscreen 12, and calculates a score for the user answering the question bank 131 according to the operation data and the questions. For example, the processing unit 11 may calculate the score by determining whether the touch locations as included in the operation data respectively match the locations on the touchscreen 12 that displayed the color according to the questions, and by determining whether each answering time is within a second predetermined time length of, for example, but not limited to, ten seconds. If both the aforementioned determinations are affirmative, the score that is calculated by the processing unit 11 may be a perfect score (e.g., a hundred points); otherwise, the processing unit 11 may deduct points from the perfect score accordingly. For example, for each location on the touchscreen 12 that displayed the color according to one of the questions but was not in contact with one of the operating pieces 31, 32, ten points may be deducted from the perfect score; alternatively, for each touch location that is incorrect as compared to the locations on the touchscreen 12 that displayed the color according to one of the questions, ten points may be deducted from the perfect score. Furthermore, ten points may be deducted from the perfect score each time the answering time exceeds the second predetermined time length. In some embodiments, the processing unit 11 generates and records the operation data each time the processing unit 11 receives the touch signal from the touchscreen 12 until the processing unit 11 determines that all of the questions in the question bank 131 have been answered, after which the processing unit 11 calculates the score according to the operation data and the questions.

After calculating the score, the processing unit 11 may control the touchscreen 12 to display a chart (e.g., a bar chart, a pie chart, an ability radar chart, etc.) according to the score calculated. The chart may present information such as a distribution of the locations of the second through holes 82 where the questions (i.e., within the question bank 131 related to the second question board 8) are answered correctly and/or incorrectly, and the answering time for each of the questions. By looking at the chart, a training status and ability of the user may be known.

The processing unit 11 is further configured to modify the question(s) of the question bank 131 based on the operation data and the score. Therefore, when the second option 62 is selected by the user again, the processing unit 11 obtains, from the storage unit 13, the question bank 131 that is related to the second question board 8 and that includes the question(s) that has been modified. The user may place the second question board 8 on the touchscreen 12 again. This time, the processing unit 11 controls the touchscreen 12 to display the color at the location(s) on the touchscreen 12 according to the question(s) that has been modified. A modification of the question(s) may include an adjustment of the location(s) on the touchscreen 12, where the location(s) on the touchscreen 12 is (are) displaying the color and correspond(s) to the position(s) of the second through holes 82 that is (are) displaying the color, an adjustment in the way of displaying the question (e.g., the touchscreen 12 displaying the color for just a few seconds, which requires the user to memorize the location(s) of the color being displayed), and/or an adjustment of the first predetermined time length and/or the second predetermined time length. In this way, the interactive rehabilitation system may adaptively provide questions that are best suited to the training needs of the user in order to help improve the user's ability and gradually assist in the upper limb rehabilitation of the user.

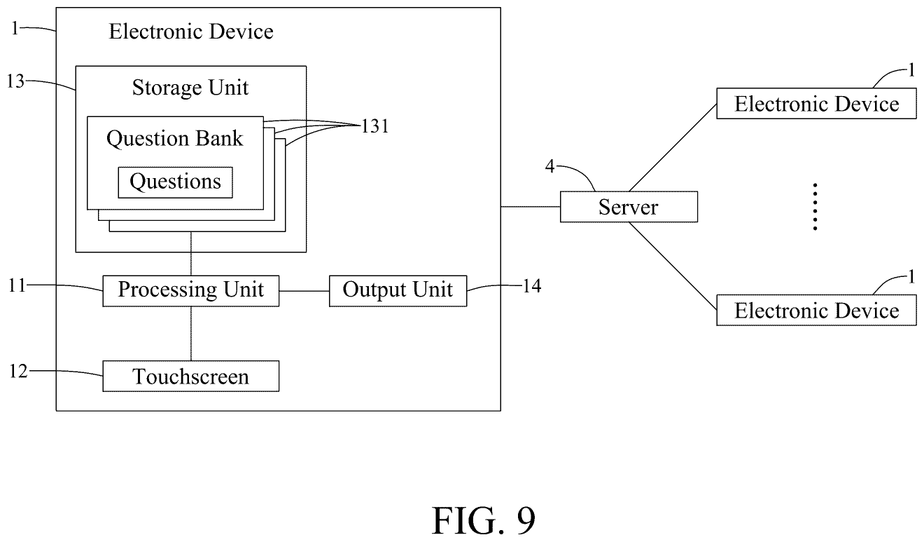

Referring to FIG. 9, the interactive rehabilitation system according to a second embodiment of this disclosure is presented. The second embodiment differs from the first embodiment in that, in the second embodiment, the interactive rehabilitation system further includes a server 4 that is communicatively connected to the electronic device 1. For example, the server 4 is a remote server, and may be connected to the electronic device 1 through a network such as the Internet, and may be communicatively connected to multiple electronic devices 1, simultaneously, in order to, for example, provide the question banks 131 to each of the electronic devices 1. The server 4 stores the question banks 131 that are related respectively to the first question board 7, the second question board 8, and the third question board 9. The following paragraphs will describe two ways in which the server 4 is able to provide the question banks 131 to each of the electronic devices 1.

In the first way, similar to the first embodiment, the processing unit 11 controls the touchscreen 12 to display the operating options that are associated respectively with the question banks 131 and that correspond respectively to the first question board 7, the second question board 8, and the third question board 9. When the processing unit 11 determines that one of the operating options is selected, the processing unit 11 generates and transmits a selection signal that corresponds to the selected one of the operating options to the server 4. In response to receipt of the selection signal from the processing unit 11, the server 4 obtains the question bank 131 that is stored within the server 4 and that corresponds to the selection signal (i.e., corresponding to the selected one of the operating options), and transmits the question bank 131 to the electronic device 1.

In the second way, each of the first, second and third question boards 7, 8, 9 includes a back side 73, 83, 93 formed with a touch pattern 70, 80, 90 that is associated with a corresponding one of the question banks 131 (i.e., the embodiments shown in FIGS. 7 and 8). When the touchscreen 12 detects the touch pattern 70, 80, 90 of one of the question boards 7, 8, 9 that is placed on the touchscreen 12, the touchscreen 12 transmits a pattern signal that corresponds to the touch pattern 70, 80, 90 to the processing unit 11. The processing unit 11 then transmits the pattern signal to the server 4. In response to receipt of the pattern signal from the processing unit 11, the server 4 obtains the question bank 131 that is stored within the server 4 and that corresponds to the pattern signal (i.e., corresponding to the one of the question boards 7, 8, 9 that is placed on the touchscreen 12), and transmits the question bank 131 to the electronic device 1.

When the processing unit 11 of the electronic device 1 starts the timer, the processing unit 11 transmits a start signal to the server 4. Then, the processing unit 11 generates and transmits the operation data to the server 4 each time the processing unit 11 receives the touch signal from the touchscreen 12 until the timer counts to the first predetermined time length, and the processing unit 11 stops receiving the touch signal. Then, the processing unit 11 transmits a stop signal to the server 4. Each of the start signal, the operation data, and the stop signal includes an identification number that is unique to and associated with the electronic device 1 and that enables the server 4 to identify which one of the electronic devices 1 the start signal, the operation data, and the stop signal are from.

The server 4, in response to receipt of the start signal, expects a continuous stream of the operation data from the electronic device 1, and the stop signal that indicates the end of the continuous stream of the operation data. In response to receipt of the stop signal from the electronic device 1, the server 4 calculates the score according to the operation data, and the questions from the question bank 131 that was transmitted to the electronic device 1. Then, the server 4 formulates the chart based on the score thus calculated, and transmits the chart and the score to the electronic device 1. The electronic device 1, in response to receipt of the chart, controls the touchscreen 12 to display the chart. By looking at the chart, the training status and ability of the user may be known.

The server 4 is further configured to modify the question(s) of the question bank 131 based on the operation data and the score. When one of the operating options is selected by the user again, the server 4 obtains the question bank 131 stored within the server 4 and including the question(s) that has been modified, and transmits the question bank 131 to the electronic device 1. The modification of the question(s) in this embodiment is similar to the modification of the question(s) in the first embodiment. Then, the processing unit 11 of the electronic device 1 controls the touchscreen 12 to display a color at the location on the touchscreen 12 according to the question(s) that has been modified. In this way, the interactive rehabilitation system may adaptively provide questions that are best suited to the training needs of the user based on training results (i.e., the training status and ability) of the user from previous sessions, in order to help improve the user's training ability and gradually assist in the upper limb rehabilitation of the user.

It should be noted that, one way of modifying the question(s) in the question bank 131 as mentioned above may be through the use of machine learning models. Specifically, by continuously providing training information that is related to the user to a machine learning model (algorithm), the machine learning model may familiarize itself with the training status and ability of the user, and may automatically modify the question(s) in the question bank 131 in order to gradually assist the user to improve the ability of the user.

In sum, the question boards 7, 8, 9 may be detachably placed on the touchscreen 12 of the electronic device 1 one at a time. Each of the question boards 7, 8, 9 is formed with the through holes 72, 82, 92, and a user (e.g., a patient) may insert one of the operating pieces 31, 32 into one of the through holes 72, 82, 92. The touchscreen 12 may transmit a touch signal to the processing unit 11 when the touchscreen 12 detects a contact from the one of the operating pieces 31, 32. The processing unit 11 then generates and records the operation data based on the touch signal thus received. The operation data includes the location where the one of the operating pieces 31, 32 is in contact with the touchscreen 12 and the time length of the take it takes for the user to place the one of the operating pieces 31, 32. The processing unit 11 evaluates the ability of the user based on the operation data in order to accurately identify and present the training and rehabilitation status of the user. By virtue of the foregoing operations of the interactive rehabilitation system according to this disclosure, a rehabilitation therapist may quickly grasp a patient's rehabilitation status, can instantly provide personalized optimal training based on the training results, and can adjust a rehabilitation plan according to the patient's needs. In addition, by virtue of connecting the server 4 to multiple electronic devices 1 at the same time, multiple people may be supported to rehabilitate at the same time, and the identification of the training and rehabilitation statuses, as well as the evaluation of the ability of the patients may also be done at the same time. This may reduce the heavy workload of the rehabilitation therapists.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An interactive rehabilitation system, comprising:
an electronic device including a processing unit, and a touchscreen that is electrically connected to said processing unit;
a question board configured to be detachably placed on said touchscreen, and including a board body that is formed with a plurality of through holes; and
an operating piece having a cross-sectional shape that matches a cross-sectional shape of one of said through holes,
wherein said touchscreen is configured to detect a contact from said operating piece, and to transmit a touch signal to said processing unit when said operating piece is inserted through one of said through holes that has the cross-sectional shape matching the cross-sectional shape of said operating piece, when said operating piece is in contact with said touchscreen, and when said touchscreen detects a contact from said operating piece,
wherein, in response to receipt of the touch signal, said processing unit is configured to generate and record operation data based on the touch signal thus received, the operation data including a location where said operating piece is in contact with said touchscreen and a time length of time it takes to place said operating piece.

2. The interactive rehabilitation system as claimed in claim 1, wherein the cross-sectional shape of each of said through holes is one of a rectangle, a triangle, a circle and a polygon, and said operating piece is a pillar.

3. The interactive rehabilitation system as claimed in claim 1, wherein the cross-sectional shape of each of said through holes is a circle, and said operating piece is a pillar.

4. The interactive rehabilitation system as claimed in claim 1, wherein said electronic device further includes a storage unit storing a question bank that is related to said question board and that includes a question,
wherein said processing unit is further configured to
control said touchscreen to display an operating option that is associated with the question bank and that corresponds to said question board,
obtain, from said storage unit, the question bank that is associated with the operating option when said processing unit determines that the operating option is selected,
control said touchscreen to display a color at a location on said touchscreen according to the question, the location on said touchscreen corresponding to a position of one of said through holes of said question board that is placed on said touchscreen, and control said electronic device to instruct a user to insert said operating piece into said one of said through holes where said touchscreen is displaying the color, and
calculate a score according to the operation data and the question.

5. The interactive rehabilitation system as claimed in claim 4, wherein said processing unit is further configured to modify the question of the question bank based on the operation data and the score.

6. The interactive rehabilitation system as claimed in claim 1, wherein said electronic device further includes a storage unit storing a question bank that is related to said question board and that includes a question, and said question board further includes a back side formed with a touch pattern that is associated with the question bank,
wherein said touchscreen is further configured to transmit, to said processing unit, a pattern signal that corresponds to said touch pattern of said question board when said touchscreen detects said touch pattern of said question board that is placed on said touchscreen,
wherein said processing unit is further configured to
obtain, from said storage unit, the question bank according to the pattern signal;
control said touchscreen to display a color at a location on said touchscreen according to the question, the location on said touchscreen corresponding to a position of one of said through holes of said question board that is placed on said touchscreen, and control said electronic device to instruct a user to insert said operating piece into said one of said through holes where said touchscreen is displaying the color, and
calculate a score according to the operation data and the question.

7. The interactive rehabilitation system as claimed in claim 6, wherein said processing unit is further configured to modify the question of the question bank based on the operation data and the score.

8. The interactive rehabilitation system as claimed in claim 6, wherein said touch pattern includes a conductive material.

9. The interactive rehabilitation system as claimed in claim 1, further comprising a server that is communicatively connected to said electronic device and that stores a question bank related to said question board and including a question,
wherein said processing unit is further configured to control said touchscreen to display an operating option that is associated with the question bank and that corresponds to said question board, and to generate and transmit a selection signal that is related to the operating option to said server when said processing unit determines that the operating option is selected,
wherein said server is configured to, in response to receipt of the selection signal from said processing unit of said electronic device, select the question bank that is related to said question board according to the selection signal, and transmit the question bank to said electronic device,
wherein said processing unit is further configured to, in response to receipt of the question bank from said server, control said touchscreen to display a color at a location on said touchscreen according to the question, the location on said touchscreen corresponding to a position of one of said through holes of said question board that is placed on said touchscreen, and control said electronic device to instruct a user to insert said operating piece into said one of said through 15 holes where said touchscreen is displaying the color, and
wherein said electronic device is configured to transmit the operation data to said server, and said server is further configured to calculate a score based on the operation data and the question.

10. The interactive rehabilitation system as claimed in claim 9, wherein said server is further configured to modify the question of the question bank based on the operation data and the score.

11. The interactive rehabilitation system as claimed in claim 1, further comprising a server that is communicatively connected to said electronic device and that stores a question bank related to said question board and including a question,
wherein said question board further includes a back side formed with a touch pattern that is related to the question bank,
wherein said touchscreen is further configured to transmit, to said processing unit, a pattern signal that corresponds to said touch pattern of said question board when said touchscreen detects said touch pattern of said question board that is placed on said touchscreen;
wherein said processing unit is further configured to transmit the pattern signal to said server,
wherein said server is configured to, in response to receipt of the pattern signal from said processing unit, select the question bank according to the pattern signal, and transmit the question bank to said electronic device,
wherein said processing unit is further configured to, in response to receipt of the question bank from said server, control said touchscreen to display a color at a location on said touchscreen according to the question, the location on said touchscreen corresponding to a position of one of said through holes of said question board that is placed on said touchscreen, and control said electronic device to instruct a user to insert said operating piece into said one of said through holes where said touchscreen is displaying the color, and
wherein said electronic device is configured to transmit the operation data to said server, and said server is further configured to calculate a score based on the operation data and the question.

12. The interactive rehabilitation system as claimed in claim 11, wherein said server is further configured to modify the question of the question bank based on the operation data and the score.

13. The interactive rehabilitation system as claimed in claim 11, wherein said touch pattern includes a conductive material.

14. The interactive rehabilitation system as claimed in claim 1, wherein said operating piece has an end portion that includes a conductive material.
